(19) United States
(12) Patent Application Publication
(10) Pub. No.: US 2018/0235398 A1
(43) Pub. Date: Aug. 23, 2018

(54) MODULAR BABY FOOD PREPARATION DEVICE

(71) Applicant: CAPSULBAR TECHNOLOGIES SYS 2014 LTD, Bitzaron (IL)

(72) Inventor: Eldad MAZIEL, Mazkeret Batia (IL)

(21) Appl. No.: 15/752,869

(22) PCT Filed: Aug. 17, 2016
(86) PCT No.: PCT/IL2016/050900

§ 371 Date: Feb. 14, 2018

Related U.S. Application Data
(60) Provisional application No. 62/205,890, filed on Aug. 17, 2015.
(60) Provisional application No. 62/205,885, filed on Aug. 17, 2015.
(60) Provisional application No. 62/205,881, filed on Aug. 17, 2015.

(51) Int. Cl. A47J 31/40; A47J 31/56; A23L 33/00; A47J 31/60; A61L 2/04

(52) U.S. Cl. CPC A47J 31/407; A47J 31/56; A47J 31/404; A61L 2202/14; A47J 31/60; A61L 2/04; A23V 2002/00; A23L 33/40

(57) ABSTRACT

The present invention relates to the field of modular baby food preparation devices and use thereof. The present invention provides a modular baby food preparation device comprising: (a) a stationary unit, comprising: (i) a water boiling tank; (ii) a water cooling system; and (iii) a water pump; (b) an optionally detachable and portable baby food dispenser, fluidly connected to said stationary unit, comprising: (i) a baby food capsule holder; and (ii) an actuator for opening said baby food capsule; (c) a computer having a processor and a memory; (d) a control panel/circuit; and (e) a display unit.

### FIELD OF THE INVENTION

The present invention relates to the field of baby food preparation devices. More specifically, the present invention relates to the field of modular baby food preparation devices.

### BACKGROUND OF THE INVENTION

In infant feeding, mother's milk is usually recommended. However, in some cases breast feeding is unsuccessful or inadvisable due to medical reasons (either the baby's or the mother's), or the mother itself consciously chooses not to breast feed. For these and other situations, different infant formulae have been developed.

Generally, infant formulae are available in powder form, concentrated liquid form, or ready to feed liquid form. Powdered infant formulae are the most popular form, primarily due to their low cost, nutritional quality, and long shelf life. The key disadvantage with powdered infant formulae is the inconvenience of preparation: The powdered formula must be carefully measured into a sterilized drinking vessel containing defined amount of water which were pre-boiled and cooled, to reconstitute the formula; the drinking vessel is then sealed and shaken to ensure the powder is properly dissolved. To avoid bacterial growth, the formula should then be consumed immediately after reconstitution. After the first use, the remaining, unused powdered formula has a very short shelf life—usually of about a month, and must be discarded if not used.

If prepared and consumed in this manner, powdered infant formulae provide a safe and nutritionally good substitute for mother's milk in the situations described above. However, primarily due to the inconvenient preparation, many parents do not prepare the formulae properly and hence expose the infant to risks of infection or other risks. For example, the water may not be boiled prior to use in which case, any pathogens in the water are fed to the infant. Alternatively, batches of the infant formula may be prepared and then stored until needed. Unfortunately, if any pathogen has contaminated the formula, it then has time to replicate. In addition, open powdered formula container might be used after the recommended period, which again raises the risk of pathogen growth within the container of said powdered formula.

In hospitals and other care facilities where infants cannot receive personal, one to one attention, the practicalities associated with preparing infant formula for large numbers of infants coupled with concerns about the risk of growth of pathogens in reconstituted formula which is not consumed for several hours have led to drastic measures. For example, some hospitals will not use any powdered products insisting on the use of individual bottles of sterilized ready to drink formula. This and other measures are obviously costly.

U.S. Pat No. 8,460,732 provides a convenient method and system of preparing a single serving of nutritional composition comprising introducing water into a sealed disposable capsule containing a unit dose of the composition and an operative opening contained within the capsule to permit draining of the resulting liquid directly from the capsule into a receiving vessel. However, mixing an infant powder formula with water in a capsule has several drawbacks, such as lump formation, possible bacteria growth and others.

On the other hand, as noted above, mixing an infant powder formula with water outside a capsule, i.e. in a bottle, has its own drawbacks, e.g., it requires using a precise amount of water at the right temperature.

Therefore, it would be advantageous to have a controlled system or device that provides pre-boiled water at a desired temperature for preparing a baby food formula by mixing powdered baby food with an accurate amount of water in a bottle (post mix) and not inside the capsule.

### SUMMARY OF THE INVENTION

The present invention provides a modular baby food preparation device 100 comprising: (a) a stationary unit 101, comprising: (i) a water boiling tank 103; (ii) a water cooling system 400; and (iii) a water pump 107; (b) a baby food dispenser 106, fluidly connected to said stationary unit 101, comprising: (i) a baby food capsule holder 406; and (ii) an actuator/piston 113 for opening said baby food capsule; (c) a computer having a processor and a memory; (d) a control panel/circuit; and (e) a display unit.

The present invention further provides a modular baby food preparation device 100 comprising: (a) a stationary unit 101, comprising: (i) a water boiling tank 103; (ii) a water cooling system 400; and (iii) a water pump 107; (b) a detachable and portable baby food dispenser 106, fluidly connected to said stationary unit 101, comprising: (i) a baby food capsule holder 406; (ii) an actuator 113 for opening said baby food capsule; (iii) a secondary water container 104; (iv) a power source; (v) a control panel/circuit 302; and (vi) a water valve; (c) a computer having a processor and a memory; (d) a control panel/circuit; and (e) a display unit. This configuration enables to disconnect said portable baby food dispenser 106 from said stationary unit 101, and use it as an independent portable unit for preparing baby food formulation without the need of the stationary unit 101.

The present invention also provides a method for preparing baby food formula, said method comprising the steps of: (a) providing a modular device 100 of the invention; (b) filling said boiling tank 103; (c) providing a capsule comprising powdered baby food formula, and placing same in the capsule holder 406 of said modular device 100; (d) placing a collection container (e.g. baby bottle) underneath said portable dispenser; and (e) activating said modular device 100.

### BRIEF DESCRIPTION OF THE DRAWINGS

FIGS. 1A-1B schematically show a perspective view of a modular baby food preparation device according to one embodiment of the invention which is connected directly to the main water pipe.

FIGS. 2A-2B schematically show a perspective view of a modular baby food preparation device according to another embodiment of the invention which has an integrated water supply unit.

FIG. 3 illustrates yet another perspective view of a modular baby food preparation device according to an embodiment of the invention.

FIGS. 4A-4B schematically show a sectioned view of a cooling unit/system according to an embodiment of the invention.

FIGS. 5A-5B schematically show a sectioned view of an hydraulic connection between a docking station and a portable baby food dispenser according to an embodiment of the invention.

FIG. 6 shows a block diagram of a control circuitry of a modular baby food preparation device according to an embodiment of the present invention.

FIGS. 7A-7C show a 3-dimentional representation of a modular baby food preparation device according to a specific embodiment of the present invention with a detachable portable baby food dispenser.

FIG. 8A-8C schematically show a front cut view of a portable sterile baby food dispenser according to yet another embodiment of the present invention.

FIG. 9A-9B schematically show a front cut view of a portable sterile baby food dispenser according to yet another embodiment of the present invention.

FIG. 10 schematically shows a front cut view of a portable sterile baby food dispenser according to yet another embodiment of the present invention.

FIGS. 11A-11B show a 3-dimentional representation of the capsule placement chamber (11A) and the control panel (11B) according to specific embodiments of the invention.

### DETAILED DESCRIPTION OF THE INVENTION

The present invention provides devices, systems and methods of preparing a single serving of a baby food nutritional composition, which are based on using a sealed (disposable or reusable) capsule containing a unit dose of the baby food composition, optionally in concentrated form, to which pre-boiled water at a desired temperature are added.

Accordingly, the present invention provides a modular baby food preparation device 100 comprising: (a) a stationary unit 101, comprising: (i) a water boiling tank 103; (ii) a cooling system 400 for cooling the boiled water, e.g. within said boiling tank 103; and (iii) a water pump for pumping the water out of said boiling tank 103, e.g. out into a vessel; (b) a baby food dispenser 106, fluidly connected to said stationary unit 101, comprising: (i) a baby food capsule holder 406; and (ii) an actuator 113 for opening said baby food capsule, said actuator 113 may be a linear actuator, and may be either electronic or mechanic, e.g. hydraulic; (c) a computer having a processor and a memory configured for controlling, e.g., the water boiling and cooling, the water pump, the actuator 113, etc.; (d) a control panel/circuit; and (e) a display unit for displaying, e.g., time, sensory data, power charging, water temperature, etc.

Examples of such cooling system 400 include, but are not limited to: air blower, thermoelectric Peltier unit, etc. In a specific embodiment, the boiled water is cooled after boiling within said boiling tank 103. In another specific embodiment, the boiled water is first transferred to a secondary cooling container for cooling. In yet another specific embodiment, the boiled water is passed through a pipe of said cooling system 400 for cooling as it exits the device 100. In a specific embodiment, the water is cooled to a temperature of from about 25° C. to about 37° C.

In certain embodiments, the user may determine the temperature of the water, such that the device 100 maintains said user-determined temperature, instead of or in addition to the option of maintaining the water according to a factory-predefined temperature.

In certain embodiments, the stationary unit 101 is designed to be used with already treated water and it may further comprise an additional micro-bacterial filter. The boiling tank 103 is adapted to connect to a portable baby food dispenser 106 through a hydraulic/fluid connector 200 and a water pump 107′.

In a specific embodiment, the baby food dispenser 106 of the modular baby food preparation device 100, is portable, and further comprises: (iii) a secondary water container 104 for holding pre-boiled water that are pumped from said boiling tank 103; (iv) a power source, optionally rechargeable; (v) a control panel/circuit 302; and (vi) a water valve for allowing the water to flow from said secondary water container 104 out of said portable dispenser 106, and to, e.g., a baby bottle.

In a specific embodiment, said secondary water container 104 comprises thermal isolation for maintaining the water therein at a predetermined temperature of from about 25° C. to about 37° C.

In a specific embodiment, said portable dispenser 106 comprises an additional water pump for aiding in exiting the water from said secondary water container 104 out of said portable dispenser 106. In a specific embodiment, said additional water pump is part of, or constitutes said water valve that allows the water to flow from said secondary water container 104 out of said portable dispenser 106.

In yet another specific embodiment, said portable dispenser 106 further comprises a mechanism (optionally electronic) that enables its activation only when positioned in an upright situation. In such a case, when the portable dispenser 106 is tilted it cannot be activated and the water conduits and valves remain lock to thereby prevent water passage and unintentional leakage.

In yet another embodiment, the stationary unit 101 comprises a boiling tank 103 with a heating element that heats water inside said tank 103 to a boiling or close to a boiling temperature, in order to sterilize the water inside. The stationary unit 101 may also comprise a temperature sensor and a cooling unit 400 for cooling the heated water to temperatures of from about 25° C. to about 37° C. The cooled water from the boiling tank 103 is then pumped by a pump 107 through a water conduit and hydraulic connector, either outside the device 100 and into, e.g., a baby bottle, or to a portable baby food dispenser 106.

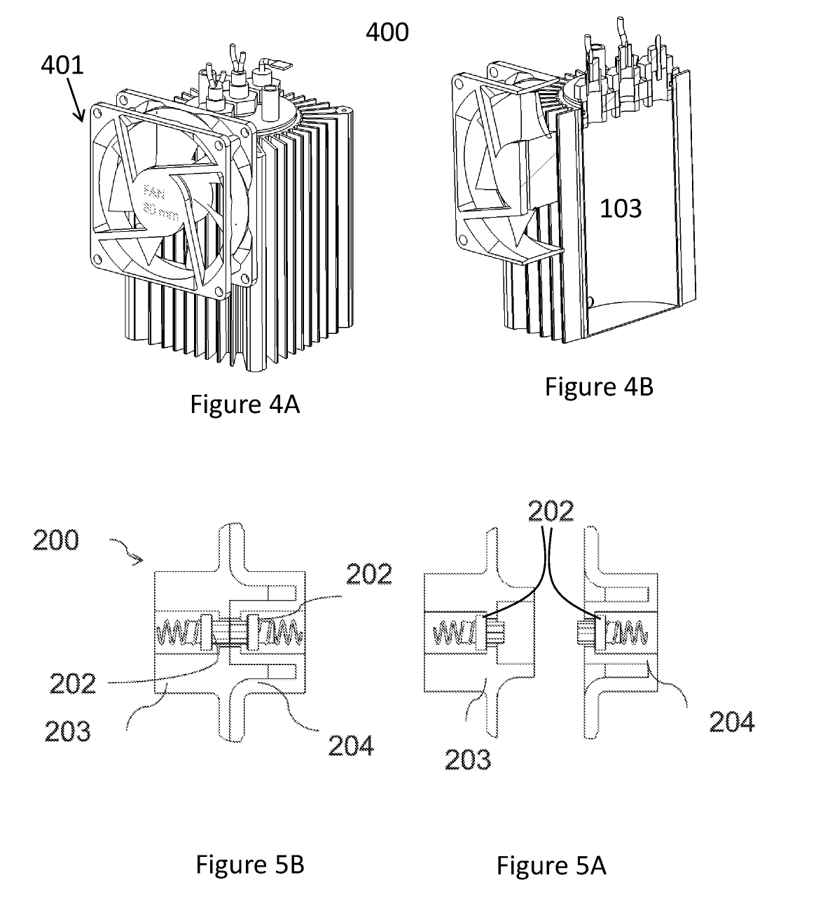

FIG. 4A illustrates a cooling fan 401 which is attached to a heat exchanger located around the boiling tank 103 and is used to cool the water inside said boiling tank 103. However, it should be noted that the cooling fan 401 may be, alternatively, attached directly to the wall of said boiling tank 103. Other alternatives for cooling the water within said boiling tank 103 include a thermoelectric cooler or a heat exchanger, or any combination thereof. When the water inside the boiling tank 103 reach the desired temperature, it is pumped by a pump 107 through a water conduit either outside the device 100 and into, e.g., a baby bottle upon activation by the user, or is pumped to a portable baby food dispenser 106, either upon activation by the user or when the level of the water within the water container 104 of said portable baby food dispenser 106 reaches a certain level (i.e. for refiling said water container 104).

In another specific embodiment, the modular device 100 of the invention further comprises a sterilization system for sterilizing the different components of said device 100, said sterilization system comprises: an activation button, optionally with a baby safety mechanism, and a CPU, designed such that upon activation, boiling water exit said boiling tank 103 and are passed through and out of the modular device 100 thereby heat sterilize it and its components.

In certain embodiments, the modular device 100 of the invention further comprises at least one sensor for measuring at least one of: water temperature, water level (in any one of the water containers/tanks), water clarity, water contaminants, battery charge level, and any other parameter that is required by the user and or authorities.

In a specific embodiment, the modular device 100 of the invention further comprises a scanner 708 or sensor for the identification of the capsule type being used/inserted into the baby food capsule holder 406. Accordingly, the device 100 may recognize the capsule being used and can calculate the amount of water that needs to add. Alternatively, the user may manually determine the amount of water to be added. In a specific embodiment, the user may use the device 100 for pouring water without preparing baby food, e.g. by pressing the activation button without placing a baby food capsule in the capsule holder 406, thereby obtaining only drinking water at a desired temperature.

In yet another specific embodiment, the modular device 100 of the invention further comprises a water sterilization mechanism, e.g. for sterilizing the water entering either the boiling tank 103 or the secondary water container 104, and/or the water exiting therefrom. Non-limiting example of such a sterilization mechanism is a filter, such as a microfilter.

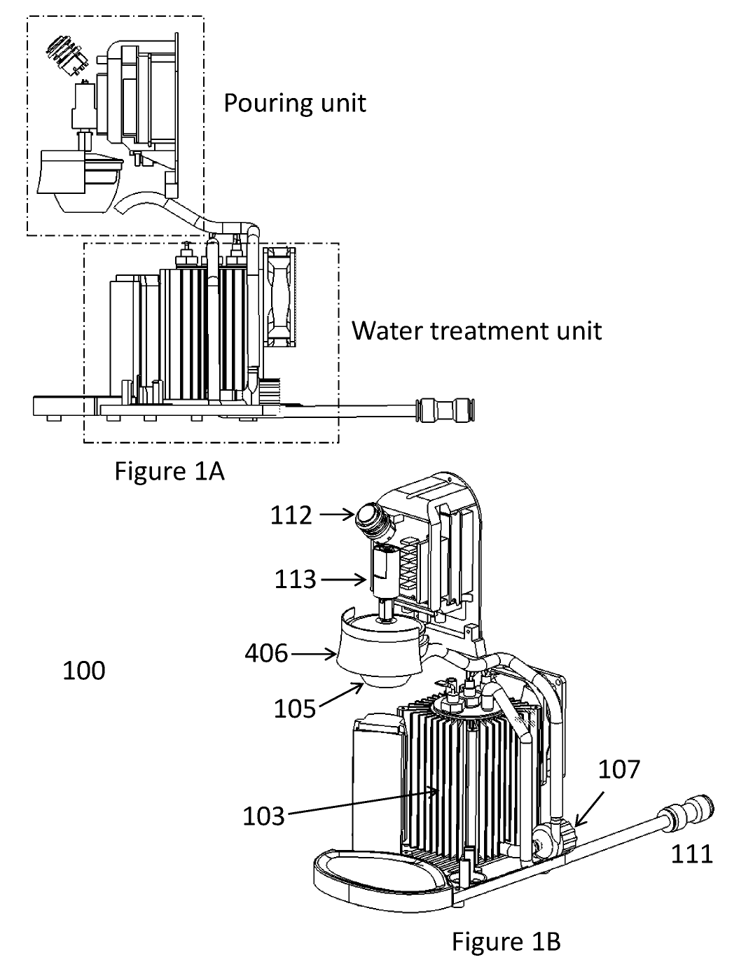

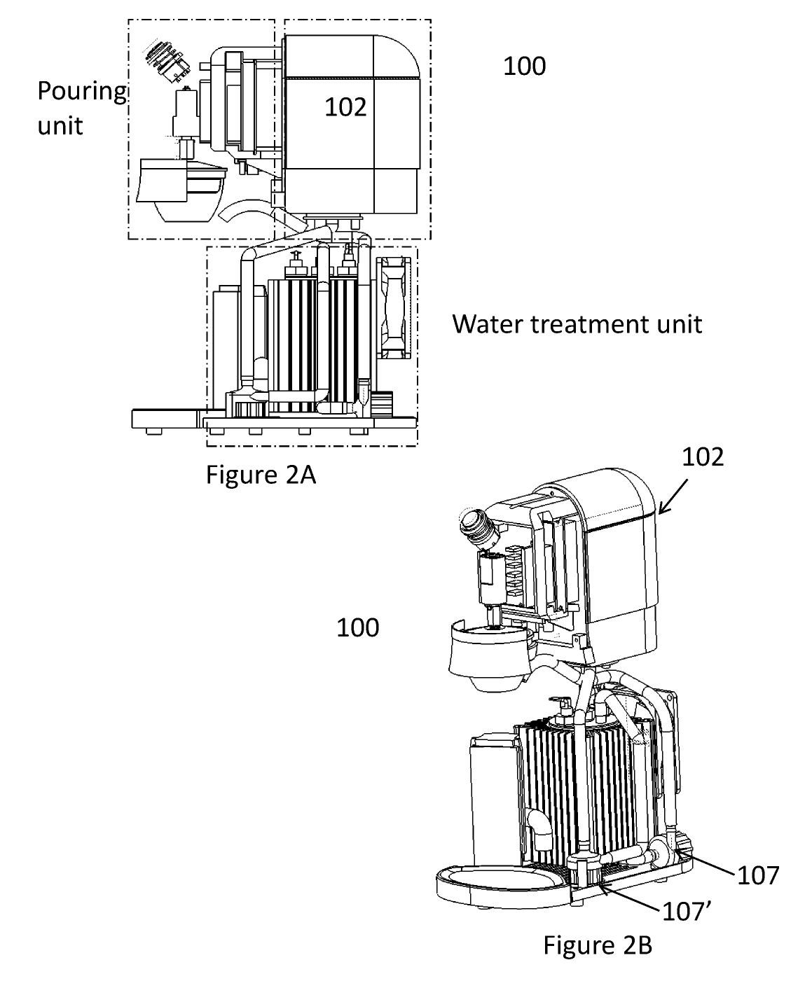

In specific embodiments, the modular device 100 of the invention further comprises a main water reservoir 102 (see FIG. 2), which is optionally detachable, instead of a direct fluid connection to a water tap (FIG. 1). In a specific embodiment, the modular device further comprises an additional water pump for pumping water from said main water reservoir 102 to said boiling tank 103. In yet another specific embodiment, the portable dispenser 106 further comprises a water heater for heating and/or boiling the water within said secondary water container 104.

In certain embodiments, when the modular device 100 comprises a water reservoir 102 and a portable baby food dispenser 106, said stationary unit 101 and said portable dispenser 106 are fluidly connected via a hydraulic/fluid connection 200, designed to allow water to pass from said main water container 102 to said secondary water container 104 of said portable dispenser 106, when the portable dispenser 106 is attached to said stationary unit 106, and prevents water leakage therefrom when disengaged. In a specific embodiment, said hydraulic connection 200 further comprises an electronic connection between said stationary unit 101 and said portable dispenser 106 for exchanging data therebetween and charging said power source od said portable dispenser 106.

In certain embodiments, the modular device 100 of the invention further comprises an air inlet 600 and an air filter, for preventing contamination of the device 100 and/or its components and/or the prepared baby food, due to contaminants in the air entering the device. Such an air inlet 600 may be connected to the water containers 102,103,104, in which the water level changes when the device 100 is activated, which requires insertion and extraction of air from said water containers.

The present invention further provides a method for the safe and convenient preparation of a liquid nutritional baby composition comprising inserting a sealed disposable capsule containing a unit dose of the composition in concentrated form into a modular device 100 as described above, the capsule may have an outlet/seal which opens in response to pressure within the capsule, wherein said pressure may be applied by, e.g., an actuator 113 located within said device 100. Once a drinking vessel is placed underneath the capsule's outlet and said device 100 is activated, the sealed capsule is opened and water is introduced into said vessel to mix with the concentrate from said capsule to form a liquid nutritional composition.

It should be noted that although the application describes and relates mainly to baby food containing capsules, the nutritional composition within such capsules may be any desired composition with and desired content. Examples of preferred nutritional compositions for use in the method and device 100 of the present invention are infant formula, growing up milks and liquid infant cereals. The ingredients of the composition are not critical to the method of the present invention and any powder or liquid concentrate may be used. Examples of different types of infant formula that may be used in the method and device 100 of the present invention include whey protein dominant formulas, formulas containing a mixture of whey and casein, formulas based on other proteins such as soy, formulas in which the protein component is partially or extensively hydrolysed, etc. It should be noted that although the application refers mainly to baby food capsules and preparations thereof, other types of preparations can be made using the device 100 of the invention, depending on the type of capsule being used and its content. For instance, capsules containing nutritional additives or protein shakes for athletes or elderly people may be used by the device 100 of the invention for preparing such supplements or food formulations.

The use of a fresh sterile capsule with the device 100 and/or according to the method of the invention for the preparation of each serving of the baby food composition, by opening each capsule just before use, pouring its content into a container, e.g. baby bottle, and adding thereto sterile water—without the need to use the water to empty the capsule, i.e. without contacting the water with the content of the capsule, greatly facilitates the safe preparation of single servings of the composition allowing individual servings of ready to drink, e.g. infant formula, to be prepared with substantially reduced or even eliminated risk of contamination from previously prepared servings or from the environment.

In a specific embodiment, a ready to drink nutritional composition may by using more than one capsule to prepare a single serving. This permits the introduction of a degree of flexibility in the compositions that may be prepared. For example, a range of capsules containing different supplements may be manufactured and consumers may be provided with instructions as to how to combine these to prepare a personalized composition suited to the particular needs of the recipient.

For a better understanding of the invention and to show how it may be carried into effect, reference will now be made, purely by way of example, to the accompanying drawings. With specific reference to the drawings in detail, it is stressed that the particulars shown are by way of example and for purposes of illustrative discussion of preferred embodiments of the present invention only, and are presented for the purpose of providing clear exemplary description of the principles and conceptual aspects of the invention. In this regard, no attempt is made to show structural details of the invention in more detail than is necessary for a fundamental understanding of the invention. From the description taken together with the drawings it will be apparent to those skilled in the art how several forms of the invention may be embodied in practice. Moreover, it is to be understood that the phraseology and terminology employed herein is for the purpose of description and should not be regarded as limiting the scope of the invention hereof.

FIGS. 1A and 1B depict a modular baby food preparation device 100 according to one embodiment of the invention. As illustrated, the device 100 is connected directly to a water source, such as a water tap. The device 100 comprises a water treatment unit which may comprise a boiling tank 103, water inlet and outlet, heater, temperature sensor, water level sensor, steam releasing vent, etc. In said water treatment unit the water is boiled and cooled within said boiling tank 103, and then transferred to a pouring unit holding the baby food capsule 105. Upon activation, e.g. pressing the activation button 112, a predefined amount of cooled water is pumped using a water pump 107 into a dedicated container, e.g. a baby bottle, whereas said capsule is opened, e.g. using an actuator 113, and the baby food formula therewithin is poured into said container. The user can then mix and prepare the final baby food formula. The order of pouring may vary—either the water is pumped first or the baby food formula is poured first. Alternatively, they may be poured simultaneously into the container.

FIGS. 2A and 2B depict a modular baby food preparation device 100 according to another embodiment of the invention. As illustrated, the device 100 comprises a dedicated water reservoir 102, which obsolete the need to be connected directly to a water source. In this configuration, the device 100 may further comprise an additional water pump: the first 107′ is to pump water from said water reservoir 102 to said boiling tank 103, and the other 107 for pumping the boiled (and optionally cooled) water from said boiling tank 103 either into the final container or to a secondary water container 104 located within said pouring unit, i.e. when the device comprises a portable baby food dispenser 106.

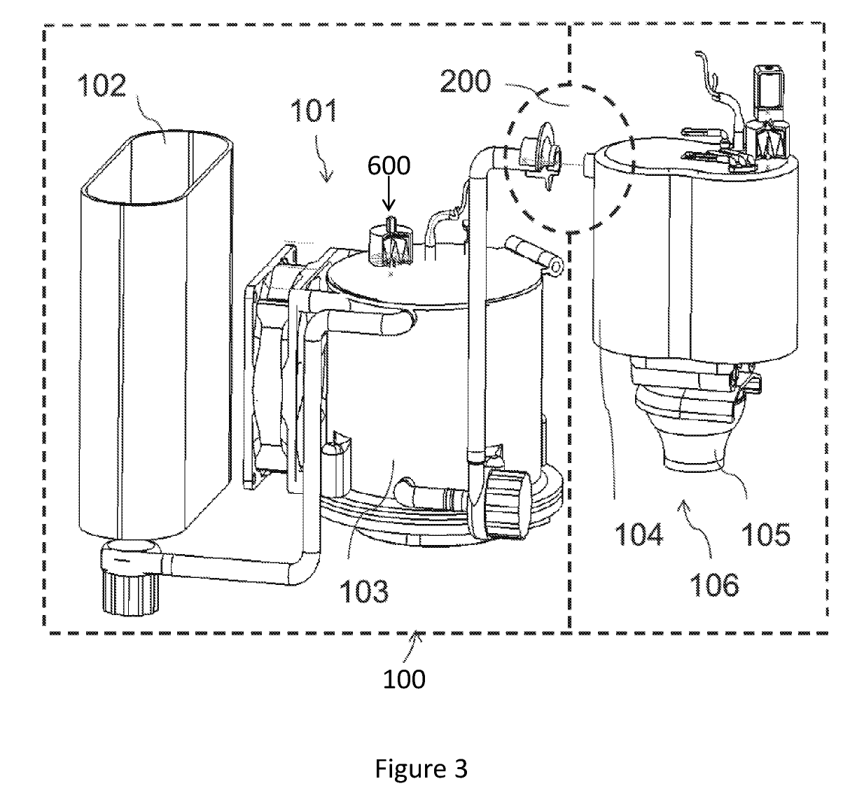

FIG. 3 depicts a modular baby food preparation device 100 according to one embodiment of the invention. As illustrated, the baby food preparation device 100 comprises a docking station 101, which is electrically and fluidly/hydraulically 200 connected to a portable baby food dispenser 106. Said docking station 101 is adapted to automatically pump pre-boiled and cooled/heated water from a boiling tank 103 into said portable dispenser 106 when it receives a low water level signal from the portable dispenser 106. The boiling tank 103 is adapted to hold heated water at 25° C.-37° C. and replenish it if and when needed, from a water reservoir 102. Notably, FIG. 3 illustrates a container of water reservoir 102, yet the device 100 may be connected directly to the tap (FIG. 1).

In certain embodiments, as illustrate in FIGS. 2 and 3, the modular baby food preparation device 100 comprises a docking station 101 and a detachable portable baby food dispenser 106, wherein the docking station 101 comprises a water reservoir 102 permanently or detachably connected through a reservoir water conduit to a boiling tank 103. The water reservoir 102 is used as a water source for said boiling tank 103, and provides the required amounts of water through a reservoir water pump 107′.

The water container 102 and/or the boiling tank 103, comprise a water level sensor that monitors the water level. They may also comprises a temperature sensor that measures the water temperature inside the container(s). The boiling tank 103 further comprises a heating element for heating and heat sterilizing water located therewithin.

As known, the preparation of baby food formula requires water at temperature of 25°-37° C. Therefore, the modular device 100 further includes a cooling system 400, such as air fan 401 attached to said boiling tank 103. Alternatively, the boiling tank 103 includes a thermoelectric cooler, or a thermoelectric heat exchanger, which receives the heated water from the boiling tank 103 through a heat exchanger inlet conduit and a heat exchanger water pump and releases it back through to the boiling tank 103 through a heat exchanger outlet conduit.

In certain embodiments, the boiling tank 103 further comprises a hydraulic/fluid connector 200 that connects it to a portable baby food dispenser 106. Boiled water from the boiling tank 103, which was cooled, is supplied through a water pump 107, a water conduit and the hydraulic connector 200 to the water container 104 of the portable baby food dispenser 106.

The boiling tank 103 further contains an air opening and a microbial filter for sterilizing the air that enters into said boiling tank 103, e.g. when water is pumped out and air enters.

In certain embodiments, and as illustrated in FIG. 3, the present invention provides a portable sterile baby food dispenser 106. The dispenser 106 comprises a water container 104, thermally insulated from the environment, e.g., by an insulator. The container 104 is adapted to receive heated water from a stationery baby food preparing unit 101, through a hydraulic connection port 200. The container 104 may also comprise a water level sensor and a water temperature sensor. In a specific embodiment, said dispenser 106 further comprise a heating element which is used to keep the temperature of the water inside the water container 104 constant, i.e. when the portable baby food dispenser 106 is not connected to said docking station 101.

In certain embodiments, the portable dispenser 106 further comprises a linear motor/actuator 113, which draws his power from said power source, e.g. rechargeable batteries. The linear motor/actuator 113, upon activation, causes a downward motion of a piston 113, which is pushed against a baby food capsule 105 held-by/placed-in a capsule receptacle/holder 406. Pushing against the capsule by said piston causes the capsule 105 to open, e.g. by an autonomous opening mechanism, and to release the baby food formula stored therein. In a specific embodiment, the portable dispenser 106 also includes an air opening and a microbial filter for removing pathogens from the air as it is drawn into the water container 104.

FIGS. 4A and 4B illustrate a possible cooling system 400 according to the invention, comprising a fan 401 associated with chilling ribs arranged around the boiling tank 103.

FIGS. 5A and 5B illustrate an enlargement of the fluid/hydraulic connection 200 between said docking station 101 on the one end, and said portable baby food dispenser 106 on the other, according to one embodiment of the invention. As illustrated, said fluid connection 200 is comprised of two mating parts: a first part 203 located at said docking station 101, and a second part 204 located at said portable dispenser 106. In a specific embodiment, before a mating connection between the two parts 203,204 is established (FIG. 5A), both parts 203,204 are hermetically sealed by at least one sealing element 202. When the fluid connection 200 is established between said parts 203,204 (FIG. 5B), said sealing element(s) 202 is retracted, thus creating a path for liquid to pass through said fluid connection 200.

The operation of the modular baby food preparation device 100 of the invention is regulated by a dedicated control circuit that may receive and store in its memory information from docking station's sensors and from sensors of the portable baby food dispenser, while it is connected to the docking station. When a portable baby food dispenser 106 is connected to the docking station 101, the docking station is adapted to automatically refill the water stored in the portable dispenser and to charge its batteries. The docking station 101 is further adapted to receive and store information that pertains to baby food preparation times, food temperature, baby food capsule composition, etc.

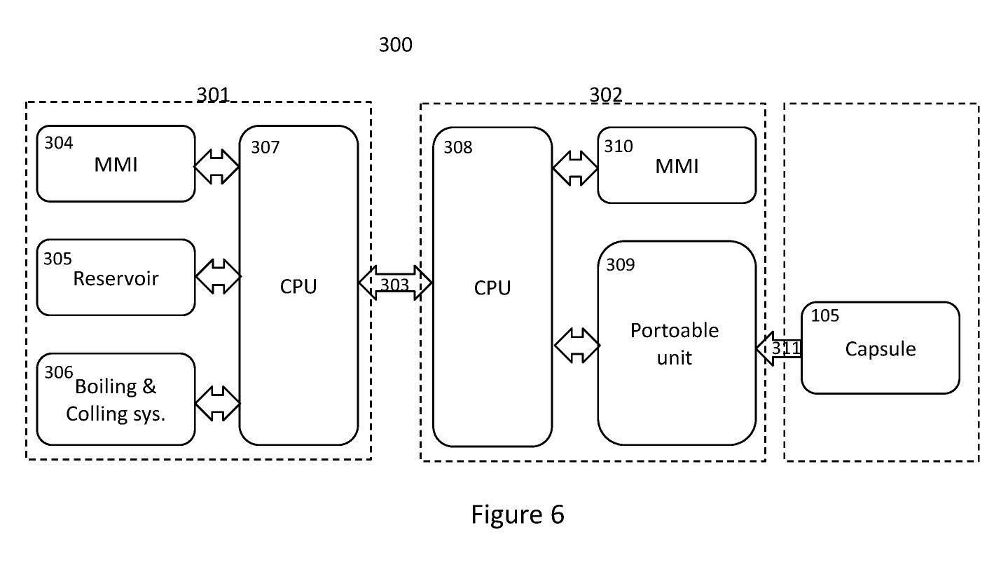

FIG. 6 illustrates one possible configuration of a block diagram of a control circuitry 300 of the modular baby food preparation device 100 according to one embodiment of the invention. The control circuitry 300 is comprised of a docking station control circuit 301 connected to a portable dispenser control circuit 302, e.g., via a data link 303.

In certain embodiments, the portable dispenser control circuit 302 comprises a portable dispenser control module 309 adapted to read an encoded data 311 located on a baby food capsule 105. The portable dispenser control module 309 is further adapted to receive data from various sensors within said preparation device 100, such as water level sensor, temperature sensor, etc., and to operate the portable dispenser 106 according to this data and the capsule's data. The docking station control circuit 301 as well as the portable dispenser control circuit 302 may also comprise a man-machine interface (MMI) module 304,310 adapted to receive commands from the end user, execute them and to display sensory and other data. Such data may include, e.g., water temperature, water level, battery charge level, etc.

As illustrated in FIG. 6, the portable dispenser control circuit 302, in certain embodiments, may further comprise a dispenser CPU module 308 adapted to receive data from other modules of the control circuit 302 as well as from the docking station control circuit 301.

As illustrated in FIG. 6, the docking station control circuit 301 comprises: (i) a reservoir control module 305 that controls water level in the water reservoir 102; (ii) a water container control circuit 306, adapted to receive sensory data from water level- and temperature-sensors, and to control accordingly the water level and temperature inside said water container, e.g. by activating a pump and/or heater/cooling unit; and (iii) a man-machine interface module 304 adapted to receive commands from the user, execute them and optionally to display sensory and other data, such as water temperature, water level, battery charge level, etc.

In specific embodiments, the docking station control circuit 301 may further comprise a docking station CPU module 307 adapted to receive data from other modules of the docking station control circuit 301 as well as from the portable dispenser control circuit 302.

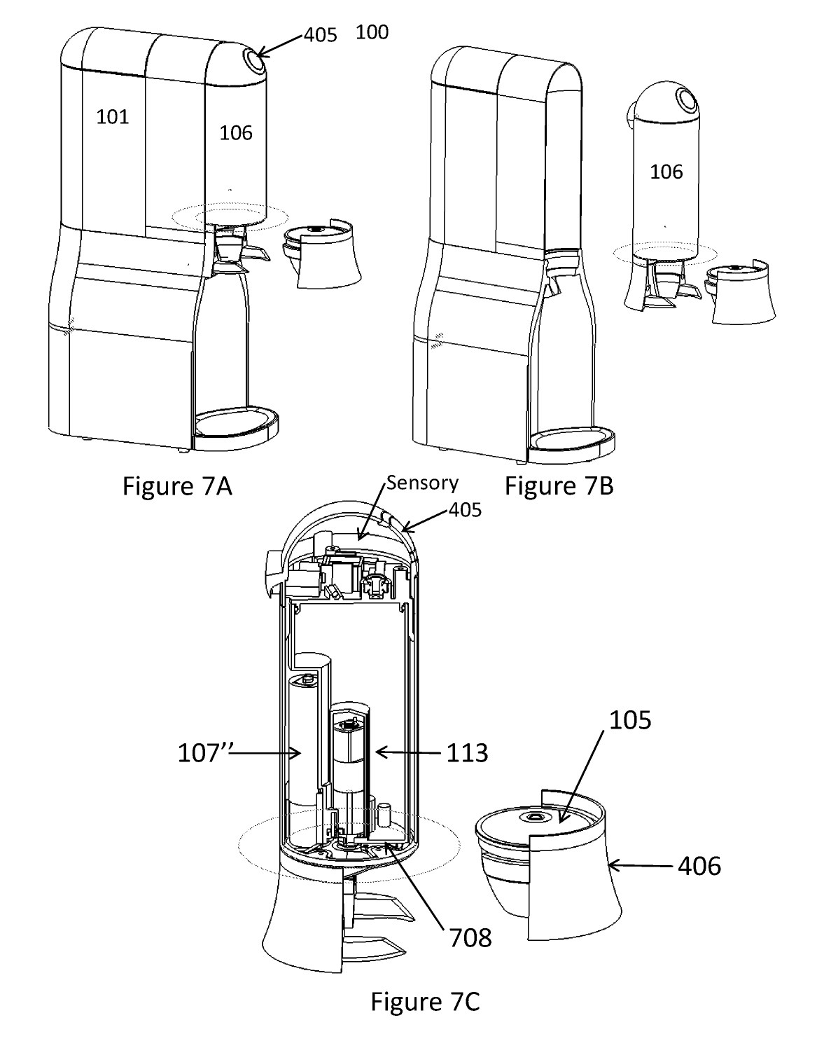

FIGS. 7A and 7B illustrate a 3-dimentional representation of a modular baby food preparation device 100 according to one specific embodiment of the present invention. The modular baby food preparation device 100 is comprised of a docking station 101 and a portable baby food dispenser 106, which is adapted to fit into a recess in said docking station 101. FIG. 7C is an enlargement and inner view of said portable baby food dispenser 106, showing the activation button 405, the actuator 113, the capsule holder/drawer 406; the inner sensory array (temperature, water level, battery charging, pressure, etc.), the capsule scanner 708, the water pump 107″, etc.

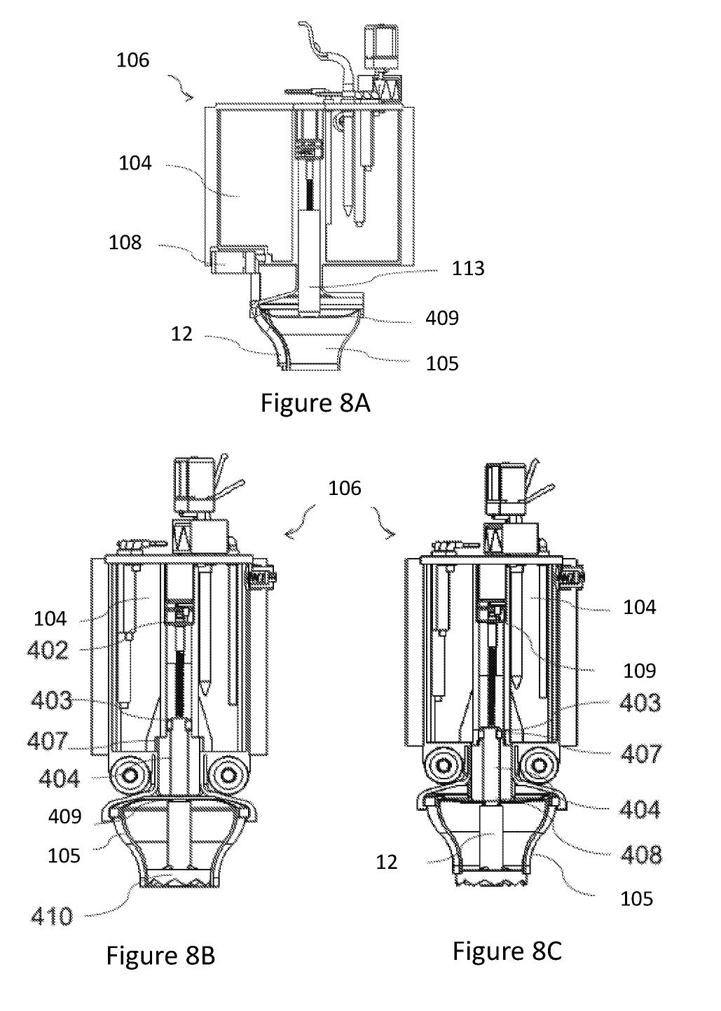

FIG. 8A illustrates a portable sterile baby food dispenser 106 according to another embodiment of the present invention. The dispenser 106 includes a water container 104 that supplies heated water to a baby bottle through a water conduit 12. The water conduit 12 is located in a grove that stretches from the top to the bottom on the side of a capsule 105. The water conduit 12 is connected at one end to a water passage lock 108 and at the other end it is adapted to fit into a bottle opening, together with the bottom end of the capsule 105. The water passage lock 108 connects the water container 104 to a water outlet leading to the water conduit 12. The water passage through the water passage lock 108 is controlled by a dedicated control circuit that opens or closes the inlet and outlet. Receiving an electrical signal from the control circuit, causes the inlet and the outlet of the water passage lock 108 to change their position from closed to open, thus allowing water from the container 104 to flow through the lock 108 and into the water conduit 12. The movement of a piston 113 downwards, drives a capsule cap 409 inwards and causes the capsule 105 to cut open and to release the powdered food stored therein.

FIGS. 8B and 8C illustrate a portable sterile baby food dispenser 106 according to yet another embodiment of the present invention. The dispenser 106 includes a water container 104 that supplies heated water to a baby bottle through a capsule water conduit 12. The capsule water conduit 12 is located at the center of a baby food capsule 105 and stretches from a capsule cap 409 to the bottom of the capsule 105. The water conduit 12 is connected at one end to a hollow piston 404 (through an opening 408 in the capsule cap 409) and at the other end it connected to the bottom part of the capsule 105. The hollow piston 404 is connected by one end to a linear motor 109 and by other end to the water conduit 12. When the hollow piston 404 reaches its lowest position, a water passage is created through a hollow piston 404, thus allowing water from the water container 104 to flow through the water container outlet 407 and into the hollow piston 404, through a hollow piston inlet 403, and into the water conduit 12 through an opening 408 in the capsule cap 409. The movement of the hollow piston 404 downwards, drives a capsule cap 409 inwards and causes the capsule 105 to open and release the powdered food stored therein.

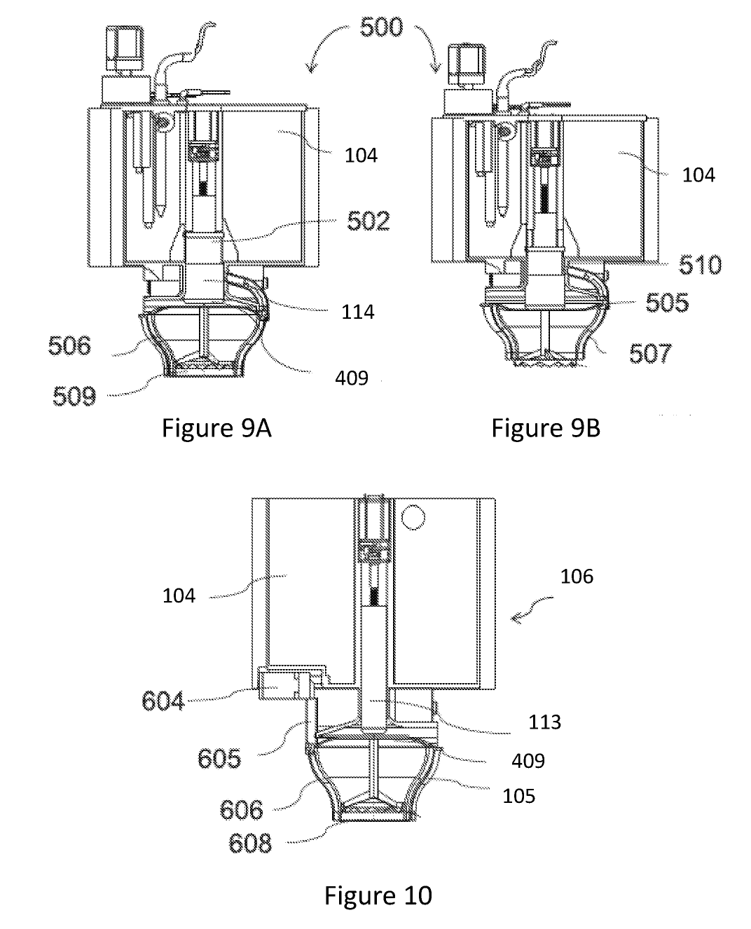

FIGS. 9A and 9B illustrate a portable sterile baby food dispenser 106 according to yet another embodiment of the present invention. The dispenser 106 includes a water container 104 that supplies heated water to a baby bottle through a dispenser water conduit and a capsule water conduit 507. The capsule water conduit 507 is formed between the walls of a double walled baby food capsule 506. The capsule water conduit 507 is connected at one end to a dispenser water conduit and at the other end it ends at the bottom 509 of the double walled capsule 506. The food dispenser 106 also comprises a water duct 510 connected at one end to a water container 104 and at other end to the dispenser water conduit. The water duct 510 is usually sealed at its ends by a piston head 114. As the piston head 114 moves down, a water passage is created through a piston narrow portion 502, thus allowing water from the container 104 to flow through the water duct 510 and to the dispenser water conduit. The movement of the piston head 114 downwards drives a capsule cap 409 inwards and causes the capsule 506 to open and release the powdered food stored therein.

FIG. 10 illustrates a portable sterile baby food dispenser 106 according to yet another embodiment of the present invention. The dispenser 106 includes a water container 104 that supplies heated water to a baby bottle through a dispenser water conduit 605 and a capsule water conduit 606. The capsule water conduit 606 is formed between two walls of a double walled baby food capsule 105. The capsule water conduit 606 is connected at one end to a dispenser water conduit 605 and at the other end it ends at the bottom 608 of the double walled capsule 105. The dispenser water conduit 605 is connected at one end to a water passage lock 604 and at the other end to the capsule water conduit 606. The water passage lock 604 is connected by a normally closed water inlet to the water container 104 and by a normally closed water outlet to the dispenser water conduit 605. The water passage through the water passage lock 604 is controlled by a dedicated control circuit that opens or closes said inlet and outlet. Receiving an electrical signal from the control circuit, causes the inlet and the outlet of the water passage lock 604 to change their position from closed to open, thus allowing water from the container 104 to flow through the lock 604 and into the water conduit 605. The movement of the piston 113 downwards drives a capsule cap 409 inwards and causes the double walled capsule 105 to open and release the powdered food stored therein.

Sterilized water can be injected into the portable dispenser by a docking station 101. The portable dispenser 106 is adapted to be attached to such docking station 101 and to receive power to activate the mobile dispenser 106 and to recharge its batteries. Alternatively, sterilized water can be poured from a kettle into the water chamber 104 of said portable dispenser 106 and the portable dispenser 106 can be recharged by a dedicated charger or by replacing its batteries.

The portable baby food formula dispenser 106 of the present invention may further comprise an information display and a memory unit. The display is used to show, e.g., the water level inside the portable baby food dispenser and its temperature. The display may also be used to show the depletion level of the rechargeable batteries.

In certain embodiments, the memory unit of the baby food formula dispenser stores information that pertains to the formula preparation, such as capsule ID, capsule content, number of meals prepared, etc. This information is further transferred and stored in the stationery unit of the baby food formula dispenser, and/or can be wirelessly transmitted to, e.g., a smartphone of the parents and/or the manufacturer of the baby food capsules, in order to maintain a steady supply of capsules to the parents.

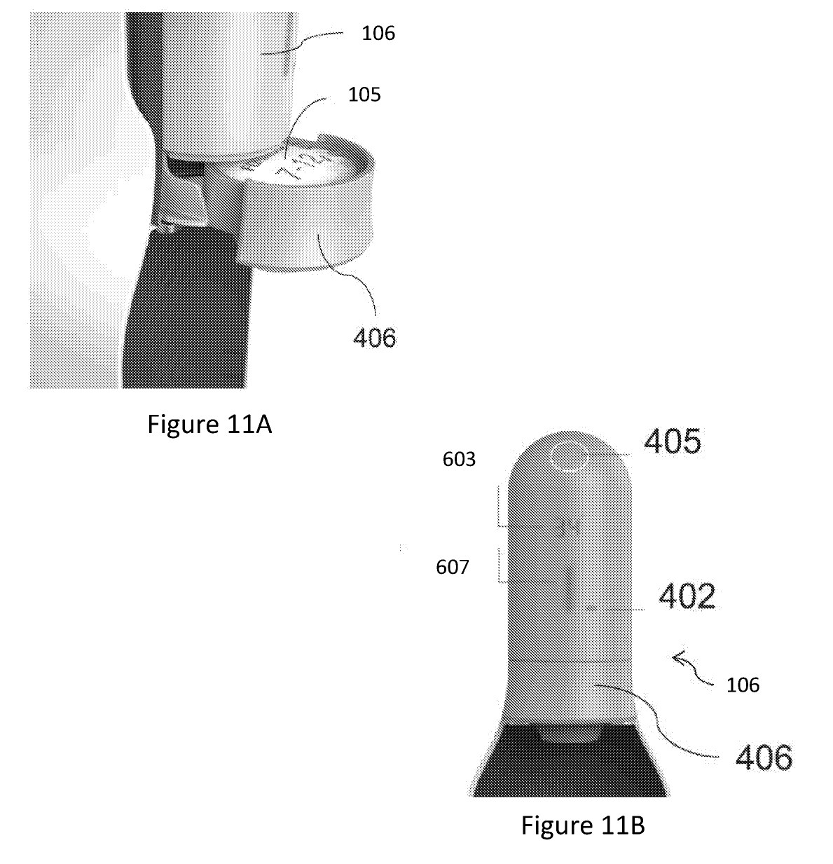

FIGS. 11A and 11B illustrate an enlargement of the 3-dimentional representation of the modular baby food preparation device 100. As illustrated, in the specific embodiments shown in FIG. 11A, the portable dispenser 106 may comprise a lower part (e.g. a drawer) 406 adapted to receive a capsule 105 comprising baby food formula powder. When said capsule 105 is placed inside said lower part 406, the lower part 406 returns/slides back into the portable dispenser 106, which then reads encoded data located on the envelope of said capsule 105 and accordingly adjusts the water amount (and optionally temperature) needed for the baby food formula preparation.

In addition, as illustrated in FIG. 11B, the portable baby food dispenser 106 may include: (i) a pushing button 405 that initiates a process that begins in releasing a controlled volume of pre-boiled and heated water into a baby bottle, optionally connected to said portable dispenser 106, followed by turning on an automatic refill of the portable dispenser 106 with pre-boiled water at a desired temperature; (ii) a water temperature indicator 603 that displays the water temperature inside the water container of said portable dispenser 106; (iii) a water level indicator 607 showing the water level inside the water container 103; and (iv) a battery charge indicator 402. IT should be noted that in certain embodiments, the user may press the pushing button 405 to pour/pump water as required, even in the absence of a capsule, e.g. for obtaining drinking water.

As mentioned above, the operation of a docking station of the present invention is regulated by a dedicated control circuitry that receives and stores in its memory information from docking station's sensors and from sensors of the portable baby food dispenser 106 while it is connected to the docking station 101. When a portable baby food dispenser 106 is connected to the docking station 101, the docking station is adapted to automatically refill the water stored in the portable dispenser 106 and to charge its batteries. The docking station 101 is further adapted to receive and store information that pertains to baby food preparation times, food temperature, baby food capsule composition, etc. This information is automatically compiled or transformed into a database and might be further wirelessly transmitted to the smartphone/tablet/computer of the parents and/or to a manufacturer of the baby food capsule, in order to maintain a steady supply of capsules to the parents.

The present invention further provides a method for preparing baby food formula, said method comprising the steps of: (a) providing a modular device 100 of the invention; (b) filling said boiling tank 103; (c) providing a capsule comprising powdered baby food formula, and placing same in the capsule holder 406 of said modular device 100; (d) placing a collection container (e.g. baby bottle) underneath, or attaching to, said portable dispenser; and (e) activating said modular device 100.

In a specific embodiment, the modular device 100 of the invention scans said baby food capsule 105 and determines the amount of water that needs to be added. Optionally, said modular device 100 further comprises a sensor that identifies the presence of such a collection container and only then activates the modular device 100. In yet other embodiments, said modular device 100 determines the water temperature upon activation, and if needed, i.e. the temperature is lower than 25° C. or higher than 37° C., activates the heating unit for heating the water, or the cooling system 400 for cooling it, respectively.

In another embodiment of the method of operating the portable sterile baby food dispenser 100 of the invention, the baby food formula is prepared at a press of a button and the preparation takes no more than a few seconds. At the beginning of the baby food preparation process, a dedicated electronic unit identifies a baby food capsule and releases a precise amount of pre-boiled and heated water into the baby bottle, followed by powder release from the capsule (or vise-versa).

1-13. (canceled)

14. A modular baby food preparation device comprising:
a) a stationary unit, comprising:
i) a water boiling tank;
ii) a cooling system; and
iii) a water pump;
b) a baby food dispenser, fluidly connected to said stationary unit, comprising:
i) a baby food capsule holder;
ii) an actuator for opening said baby food capsule;
c) a computer having a processor and a memory;
d) a control panel/circuit; and
e) a display unit.

15. The modular device of claim 14 further comprising a sterilization system for sterilizing the different components of said modular device, said system comprising: an activation button and a CPU, designed such that upon activation, boiling water exit said boiling tank and are passed through the modular device thereby heat sterilize it.

16. The modular device of claim 14 further comprising at least one sensor for measuring at least one of: water temperature, water level (in any water container/tank), water clarity, water contaminants, and battery charge level.

17. The modular device of claim 14 further comprising a scanner for the identification of the capsule type being used.

18. The modular device of claim 14 further comprising water sterilization mechanism.

19. The modular device of claim 14 further comprising a main water reservoir.

20. The modular device of claim 14 further comprising an additional water pump.

21. The modular device of claim 14, further comprising an air inlet and an air filter in said water tanks.

22. The modular baby food preparation device of claim 14, wherein said baby food dispenser is portable, and further comprising:
iii) a secondary water container;
iv) a power source;
v) a control panel/circuit; and
vi) a water valve.

23. The modular device of claim 22 further comprising at least one of: (i) a sterilization system for sterilizing the different components of said modular device, said system comprising: an activation button and a CPU, designed such that upon activation, boiling water exit said boiling tank and are passed through the modular device thereby heat sterilize it; (ii) at least one sensor for measuring at least one of: water temperature, water level (in any water container/tank), water clarity, water contaminants, and battery charge level; (iii) a scanner for the identification of the capsule type being used; (iv) a water sterilization mechanism; (v) a main water reservoir; (vi) an additional water pump; and (vii) an air inlet and an air filter in said water tanks.

24. The modular device of claim 22, wherein said power source is rechargeable.

25. The modular device of claim 22, wherein said stationary unit and said portable dispenser are fluidly connected via a hydraulic/fluid connection, designed to allow water to pass from said main water container to said secondary water container when the portable dispenser is attached to said stationary unit, and prevents water leakage therefrom when disengaged.

26. The modular device of claim 25, wherein said hydraulic connection further comprises an electronic connection between said stationary unit and said portable dispenser for exchanging data therebetween and charging said power source.

27. A method for preparing baby food formula, said method comprising the steps of:
a) providing a modular device that includes:
i. a stationary unit, having a water boiling tank, a cooling system, and a water pump,
ii. a baby food dispenser, fluidly connected to said stationary unit, having a baby food capsule holder and an actuator for opening said baby food capsule,
iii. a computer having a processor and a memory,
iv. a control panel/circuit, and
v. a display unit;
b) filling said boiling tank;
c) providing a capsule comprising powdered baby food formula, and placing same in the capsule holder of said modular device;
d) placing a collection container underneath said portable dispenser; and
e) activating said modular device.
